(19) United States
(12) Patent Application Publication
(10) Pub. No.: US 2026/0364300 A1
(43) Pub. Date: Nov. 19, 2026

(54) NO DELIVERY APPARATUS WITH CONTINUOUS ANALYSIS OF AMBIENT AIR

(71) Applicant: INOSYSTEMS, ANTONY (FR)

(72) Inventors: Frederic MARCHAL, ANTONY (FR); Yann BLANDIN, ANTONY (FR); Nathan PROUVEZ, ANTONY (FR)

(21) Appl. No.: 19/562,050

(22) Filed: Mar. 10, 2026

(30) Foreign Application Priority Data
May 15, 2025 (FR) 2505182

(51) Int. Cl. A61M 16/12; A61M 16/00; A61M 16/08; A61M 16/10

(52) U.S. Cl. CPC A61M 16/125; A61M 16/0003; A61M 16/0057; A61M 16/024; A61M 16/085; A61M 16/104; A61M 2202/0208; A61M 2202/0275; A61M 2205/3327; A61M 2205/505

(57) ABSTRACT

The invention relates to an NO delivery apparatus (1) for carrying and injecting an NO/nitrogen gas mixture into a patient circuit (2); a primary gas-analysis line (5) for measuring NO and/or NO₂ contents in the combined gas flow supplied to the patient; a secondary gas-analysis line (20) communicating with atmosphere for measuring NO and/or NO₂ contents in atmospheric air; gas-pumping means (7) for making gas flow through the primary or secondary analysis line (5; 20); control means (8) for determining NO and/or NO₂ content based on measurements within the primary or secondary analysis line (5; 20); and display means (9) for displaying the NO and/or NO₂ contents.

### CROSS-REFERENCE TO RELATED APPLICATIONS

This patent application claims priority to French Patent Application No. 2505182, filed May 15, 2025 and the entire contents of which are incorporated herein by reference.

### TECHNICAL FIELD

The invention relates to an NO delivery device or apparatus, i.e. an apparatus for supplying an NO-containing gas, intended to be connected between a (or more than one) source(s) of pressurized gaseous NO, such as a (or more than one) NO/N₂ cylinder(s), and the patient circuit, which is fed by a medical ventilator, said NO delivery apparatus comprising gas-analysing means for measuring the NO and/or NO₂ content in the combined gas flow fed to the patient or, according to the circumstances, in ambient air, i.e. air in the place where the NO delivery apparatus is found, such as a room, hall, vehicle interior or the like, and display means for displaying the corresponding NO and/or NO₂ values; and to a machine for administering gas to a patient comprising such an NO delivery apparatus.

### BACKGROUND

Nitric oxide or NO is commonly used to treat various medical conditions, as described particularly by EP-A-560928, EP-A-1516639 and US-A-10,201,564. In general, a gaseous mixture of NO (i.e. a few ppm vol.) and nitrogen (N₂) is injected by an NO delivery device into an oxygen-containing gas flow, such as an N₂/O₂ mixture, or air, or even pure oxygen, that is carried by the patient circuit of a gas-supplying machine, such as a medical ventilator, supplying the oxygen-based gas flow to said patient circuit, and the combined gas (NO/N₂/O₂) mixture thus obtained, which contains NO, oxygen and nitrogen, is then administered by inhalation to the patient to be treated having need therefor, either invasively via a tracheal tube, or non-invasively via a mask or the like, as described in US-A-5,558,083.

To ensure that the combined NO/N₂/O₂ gas flow does not contain any or only a negligible amount of toxic species, i.e. NO₂ compounds resulting from untimely oxidation of some of the NO by the O₂, the NO delivery device comprises a gas-sampling line allowing a small amount, i.e. a sample, of the final gas mixture (NO/N₂/O₂) to be drawn from the respiratory circuit, as described in US2006/207594 or EP4537879. These gas samples are then analysed within an analysis line of the NO delivery apparatus to check whether the composition of the final NO/N₂/O₂ gas flow is as desired, and particularly its NO content, which must be less than 80 ppmv, but above all its NO₂ content, which must be as low as possible, and typically less than 5 ppmv, because NO₂ is a toxic and irritating gas that may cause serious injury to the patient, even at low concentrations.

The problem is that medical personnel, i.e. doctors, nurses or the like, or paramedics may be exposed to these potentially harmful and irritating gases, i.e. NO and NO₂, in a number of ways in hospitals or transport vehicles because these gases end up in the ambient air of enclosed places or spaces where NO treatment machines are employed, such as rooms, hospital operating theatres or resuscitation rooms, corridors or the like, but also in the interiors of transport vehicles, in particular emergency vehicles, such as ambulances, helicopters, aeroplanes, etc.

These NO and NO₂ compounds accumulate in the ambient air around the NO-supplying machine under various circumstances and may be inhaled by caregivers in an untimely and/or unavoidable manner, i.e. unintentionally.

Thus, when hoses connecting NO/N₂ cylinders to the NO delivery device are purged, typically during replacement of an NO cylinder or start-up of the device, some of the gas, i.e. of the NO/N₂ mixture, contained in the flexible pipe or conduit is discharged via a discharge tube and ends up in the hospital setting or the interior of the transport vehicle.

Likewise, the gases exhaled by the patient also contain residual NO, which is released to atmosphere through the exhalation valve of the ventilator.

Moreover, analogously, NO may also be found in the ambient atmosphere around the patient in the case of untimely disconnection of the ventilator or of the patient circuit, and/or in the case of non-invasive ventilation (NIV) by mask or with a high flow rate through nasal cannulae.

In addition, some of the NO found in the atmosphere of the hospital setting or in the interior of a transport vehicle inevitably oxidizes into NO₂ when it makes contact with oxygen present in the air.

As the compounds NO and above all NO₂ are toxic to medical or paramedical personnel, maximum exposure values have been set by health authorities. Thus, in France, the occupational exposure limit (OEL) is set at 2 ppm for NO and 0.5 ppm for NO₂ for an exposure of 8 hours, and at 1 ppm for short-term exposure, for example 15 minutes.

One issue is that some caregivers, mainly paramedical personnel, fear receiving a significant exposure to NO and NO₂ whilst treating a patient with NO, this causing them anxiety and possibly moreover affecting their health, particularly if maximum exposure limits are exceeded.

Personal protective equipment for monitoring NO and NO₂ is available, but such equipment is mainly intended for use in an industrial setting because they are not or not very suitable for use in hospitals and/or in the interior of a transport vehicle, i.e. equipment not always available and/or functional, caregiving-team shift rotation, lack of knowledge by caregivers of their existence and/or their place of storage, complexity of using them in emergency vehicles, etc.

Hence, the problem addressed is that of determining the concentration of potentially toxic compounds, in particular the compounds NO and/or NO₂, in the ambient air of a hospital setting or the interior of a transport vehicle in which an NO delivery apparatus and/or a (or more than one) NO-supplying machine(s) is located and/or used, i.e. places such as a treatment room, resuscitation room, ambulance interior, helicopter interior, aeroplane interior or the like, so as to decrease the anxiety of caregivers and above all to preserve their health by allowing them to ensure that they are not being subjected to excessive toxic doses, i.e. doses typically higher than the occupational exposure limit (OEL) or short-term occupational exposure limit (ST-OEL).

### SUMMARY

The solution according to the invention is an NO delivery device or apparatus, i.e. an apparatus for supplying an NO-containing gas, typically an NO/nitrogen mixture, comprising:
a primary gas circuit for carrying an NO-containing gas, typically an NO/N₂ mixture, to at least one gas outlet configured to be fluidically connected to a patient circuit carrying an oxygen-containing respiratory gas, such as air or an oxygen/nitrogen mixture,
a primary gas-analysis line comprising first concentration-measuring means configured to take measurements of NO and/or NO₂ content (i.e. concentration) in the gas flow present within said primary gas-analysis line,
gas-pumping means configured to pump gas and make it flow through at least the primary gas-analysis line,
control means configured to process the concentration measurements taken by the concentration-measuring means and to deduce therefrom at least one NO and/or NO₂ content, and
display means configured to display said at least one NO and/or NO₂ content measured by said first concentration-measuring means.

In addition, according to the invention, the apparatus further comprises:
a secondary gas-analysis line fluidically communicating with ambient atmosphere,
the gas-pumping means are configured and/or arranged to pump ambient air and make it flow through the secondary gas-analysis line,
the secondary gas-analysis line comprises second concentration-measuring means configured to take measurements of NO and/or NO₂ content (i.e. concentration) in the ambient-air flow present within said secondary gas-analysis line,
the control means are further configured to process the concentration measurements taken by the second concentration-measuring means,
and the display means are further configured to display said at least one NO and/or NO₂ content measured by said second concentration-measuring means.

Depending on the embodiment in question, the apparatus of the invention may have one or more of the following features:
the secondary gas-analysis line is fluidically connected to the primary gas-analysis line;
the secondary gas-analysis line communicates fluidically with ambient atmosphere, via an ambient-air inlet port;
the secondary gas-analysis line is fluidically connected to the primary gas-analysis line, upstream of the gas-pumping means;
the first and/or second concentration-measuring means are configured to take measurements of NO and NO₂ content (i.e. concentration);
the secondary gas-analysis line is fluidically connected to the primary gas-analysis line between the first concentration-measuring means and the gas-pumping means;
the control means comprise control electronics, preferably control electronics comprising (at least) a microprocessor or a microcontroller;
the secondary gas-analysis line and the primary gas-analysis line are in permanent fluidic communication with each other so that the gas-pumping means simultaneously pump gas from said primary (i.e. combined NO/N₂/O₂ gas mixture) and secondary (i.e. ambient air) gas-analysis lines;
the gas-pumping means comprise a suction pump;
the suction pump comprises a gas inlet fluidically connected to the primary gas-analysis line and/or to the secondary gas-analysis line, and preferably to the primary and secondary gas-analysis lines;
the suction pump comprises a gas inlet fluidically connected to the primary gas-analysis line and the secondary gas-analysis line is fluidically connected to the primary gas-analysis line, upstream or at said gas inlet of the pump;
the first and second NO and/or NO₂ sensors take measurements of NO and/or NO₂ contents simultaneously, i.e. concomitantly, within the primary gas-analysis line (i.e. the line carrying the combined NO/N₂/O₂ gas mixture) and secondary gas-analysis line (i.e. the line carrying ambient air);
the display means are configured to display simultaneously, i.e. at the same time, and/or continuously, i.e. without interruption over time, the NO and/or NO₂ contents measured by said first and second concentration-measuring means, so as to allow the user (i.e. caregiver) to see, at any time, the NO and/or NO₂ concentration in the combined gas flow sent to the patient and in the ambient air of the setting where the NO delivery apparatus is located;
the gas-pumping means comprise a gas outlet fluidically connected to atmosphere, preferably via an outlet or vent;
the control means are configured to control the gas-pumping means to pump gas;
the gas-pumping means allow gas to be pumped at a given pumping rate between 50 and 200 mL/min;
the first and/or second concentration-measuring means comprise an NO sensor and/or an NO₂ sensor;
the display means comprise a display screen;
the screen is a touch-sensitive screen, i.e. a screen with a touch panel;
in an alternative embodiment, the NO and/or NO₂ contents measured by the second concentration-measuring means are determined and/or displayed only at the request of the user, for example after pressing an area of the touch-sensitive screen, for example a dedicated selection key or the like. In this case, pressing the dedicated key causes a valve or any similar means controlling flow of the fluid through the secondary gas-analysis line to open, or alternatively measurements taken by the second concentration-measuring means that were, for example, ignored until then to be taken into account;
the touch-sensitive screen is configured to display the NO content and/or NO₂ content determined by the first and/or second concentration-measuring means;
the primary and secondary gas-analysis lines comprise conduits, ducts, pipes, passages, tubing or the like;
the control means are configured to determine the NO and NO₂ contents by processing the measurements taken by the first and/or second concentration-measuring means.

Depending on the embodiment in question, the apparatus of the invention may also have one or more of the following additional features:
the control means are configured to control the gas-pumping means, typically the suction pump, to pump gas from the primary gas-sampling line and simultaneously from the secondary gas-sampling line;
the primary gas-sampling line comprises a non-return device;
the secondary gas-sampling line comprises a filtering means, such as a HEPA filter or the like;
the secondary gas-sampling line comprises a device with a calibrated orifice, preferably arranged between the filtering means and the site of connection of the secondary line to the primary gas-sampling line;
the primary gas-analysis line comprises one or more pressure taps and/or a device with a calibrated orifice;
the primary gas-analysis line comprises two pressure taps and a device with a calibrated orifice, the pressure taps being upstream and downstream of the device with a calibrated orifice;
the given pumping rate of the suction pump is less than or equal to 400 mL/min, preferably at least 50 mL/min, preferably between 100 and 350 mL/min, and more preferably between 200 and 300 mL/min;
the gas-pumping means comprise a diaphragm suction pump, also called a diaphragm pump, or a piezoelectric suction pump, i.e. a pump that exploits, during its operation, a dynamic piezoelectric effect resulting from an excitation at frequency;
the first and/or second means for measuring NO and/or NO₂ concentration comprise electrochemical sensors;
the first concentration-measuring means further comprise an oxygen sensor, typically an electrochemical O₂ sensor;
it comprises electrical supply means for supplying electric current to the various elements or components of the apparatus that need it in order to operate, for example to the control means, to the sensors, to the suction pump, etc. Preferably, the electrical supply means comprise a connection to the (110/220V) mains and/or a rechargeable battery or other battery, and optionally a current converter.
the control means are configured to process the measurements coming from the first and/or second concentration-measuring means with a view to deducing therefrom values of the NO and/or NO₂ concentration or content in the analysed gas flow;
the display means are controlled by the control means to display the NO and NO₂, and possibly O₂, concentrations, i.e. contents;
the display means are configured to effect one or more displays on the display screen;
the display screen displays in colour or in black and white, but preferably in colour;
the primary gas circuit of the NO delivery apparatus comprises flow-rate control means for controlling the flow rate of gas in the primary gas circuit carrying the NO/N₂ mixture, and preferably at least one solenoid valve, particularly of the proportioning type;
the primary gas circuit comprises one or more gas passages, such as gas conduits or the like;
the primary gas circuit comprises at least one gas inlet through which the NO-containing gas, typically an NO/N₂ mixture, may enter the primary circuit of the apparatus;
the gas outlet through which the NO/N₂ mixture exits the primary circuit is connected to the patient circuit by a flexible pipe or the like;
the flow-rate control means are controlled by the control means in such a way as to allow or prevent any flow of gas through the primary gas circuit; in particular, said at least one solenoid valve is controlled by the control means (i.e. piloted solenoid valve);
the primary gas circuit further comprises pressure-controlling means, in particular a gas expansion device;
the control means comprise at least one microprocessor or microcontroller arranged on at least one circuit board;
said at least one microprocessor or microcontroller implements at least one algorithm;
at least one algorithm is configured to determine NO and/or NO₂, or even O₂, contents from measurements taken by the first and/or second concentration-measuring means;
it comprises an external shell or carcass;
it comprises storage means, for example a flash memory or the like;
the values of NO, NO₂ and/or O₂ contents may be stored by the storage means.

Moreover, the invention also relates to a machine for administering gas to a patient, i.e. a machine dedicated to supplying a gas mixture containing NO and oxygen to a patient, i.e. a person, having a need therefor, comprising: the NO delivery apparatus according to the invention; a medical ventilator, such as a resuscitation ventilator; and the patient circuit to which are fluidically connected the NO delivery apparatus and the medical ventilator, and wherein the patient circuit is fluidically connected to valve means, via a primary gas-sampling line.

Depending on the embodiment in question, the gas-supplying machine of the invention may have one or more of the following features:
the medical ventilator is a ventilatory assistance device, such as a resuscitation ventilator;
the medical ventilator supplies the patient circuit with a respiratory gas containing oxygen, typically at least about 20 vol. % oxygen, preferably at least about 20 vol. % oxygen, in particular air or an N₂/O₂ mixture;
the NO delivery apparatus is fed with NO-containing gas, in particular an NO/N₂ mixture, by a (or more than one) source(s) of gas containing gaseous NO;
the (or the more than one) gas source(s) contain(s) an NO/N₂ mixture containing between 100 and 2500 ppm by volume (ppmv) of NO, the remainder being nitrogen, and preferably less than 1500 ppmv, the remainder being nitrogen, and more preferably less than 1000 ppmv, the remainder being nitrogen;
preferably, the gas source contains an NO/N₂ mixture containing from 250 to 900 ppm by volume of NO, the remainder being nitrogen, and for example of the order of 800 ppm by volume of NO, the remainder being nitrogen;
the (or the more than one) gas source(s) is a (or more than one) pressurized gas cylinder;
the patient circuit comprises an inhalation branch and an exhalation branch;
the NO delivery apparatus, the medical ventilator and the primary gas-sampling line of the NO delivery apparatus are fluidically connected to the inhalation branch of the patient circuit;
the inhalation branch and exhalation branch are connected at a junction part, such as a Y-shape part;
the patient circuit, in particular the inhalation branch, feeds a respiratory interface, such as a tracheal tube or a respiratory mask;
a flow-rate sensor is arranged in the patient circuit, preferably in the inhalation branch;
the flow-rate sensor is arranged upstream of the injection site of the gaseous NO, i.e. of the NO/N₂ mixture, coming from the NO delivery apparatus;
a gas humidifier is arranged in the inhalation branch, preferably downstream of the injection site of the gaseous NO, i.e. of the NO/N₂ mixture, coming from the NO delivery apparatus;
the patient circuit is fluidically connected to an outlet port of the medical ventilator in order to collect and transport the oxygen-containing gas delivered by the medical ventilator, such as air or an N₂/O₂ mixture;
the (or the more than one) pressurized gas cylinder(s) contain, when full, an NO/N₂ gas mixture at a pressure of at least 150 to 200 bar abs, or even at least 250 to 300 bar abs;
the patient circuit comprises flexible pipes or conduits;
the flow-rate sensor is electrically connected to the control means in order to supply them with measurements of the flow rate of the gas flow coming from the medical ventilator;
the primary gas-sampling line of the NO delivery apparatus is fluidically connected to the patient circuit at a gas-sampling site located downstream of the primary flow-rate sensor, and preferably downstream of the gas humidifier;
the gas circuit of the NO delivery apparatus is fluidically connected to the patient circuit via a gas-injection line, such as a flexible pipe;
the gas-sampling line is fluidically connected to the patient circuit via a sampling conduit, such as a flexible pipe;
the sampling conduit is connected to the patient circuit by means of a connecting part, such as a T-shaped connector, allowing gas to be sampled within the patient circuit, i.e. in the inhalation branch;
the NO delivery apparatus feeds the patient circuit with an NO/N₂ mixture, so as to dilute the gaseous NO (i.e. the NO/N₂ mixture) in the oxygen-containing flow of respiratory gas, such as air or an O₂/N₂ mixture, and obtain a final gas mixture to be administered to the patient;
the final gas mixture essentially contains nitrogen (N₂), oxygen (O₂) at a concentration of at least about 20 vol. %, and NO in a content between 1 and 80 ppmv, and typically of the order of 5 to 40 ppmv, corresponding to a desired dosage, i.e. it is an NO/O₂/N₂ gas mixture;
the final gas mixture may comprise NO₂ species resulting from oxidation of some of the NO, preferably less than 2 ppmv of NO₂, and more preferably less than 1 ppmv of NO₂.

### Definitions

In general, within the context of the invention:
“ppmv” means parts per million by volume;
“vol. %” means percentage by volume;
“NO” denotes nitric oxide;
“NO₂” denotes nitrogen dioxide;
“N₂” denotes nitrogen;
“O₂” denotes oxygen;
pressures are expressed in bar absolute, abbreviated “bar”;
the terms “concentration”, “quantity”, “proportion”, “dose” and “content” are considered to be equivalent and interchangeable;
the term “setting” should be interpreted broadly. It may designate a room, hall, bedroom, corridor or the like in a medical establishment (i.e. hospital, clinic or the like) but also the interior or a passenger compartment or internal space of a transport vehicle, such as an ambulance, helicopter, aeroplane or the like;
the terms “means of/to/for” are considered to be wholly equivalent to and interchangeable with the terms “device of/to/for”, for example the terms “control means” may be replaced by “control device”, the terms “valve means” may be replaced by “valve device”, and “storage means” may be replaced by “storage device”, etc.
by “pressure measurement”, what is meant is a pressure value (e.g. a numerical value) or a signal representative of such a pressure value, reflecting or corresponding to the gas pressure measured by a pressure sensor or the like;
by “measurement of the content of compound A”, what is meant is a value of the concentration of compound A (e.g. a numerical value) or a signal representative of such a value, reflecting or corresponding to the concentration of compound A in an analysed gas flow;
the terms “upstream” and “downstream” are used in relation to the normal direction of flow of the gas, namely in the direction leading towards the patient.

### BRIEF DESCRIPTION OF VIEWS OF DRAWINGS

The invention will now be better understood by virtue of the following detailed description, which is provided by way of non-limiting illustration, with reference to the appended figures, in which:

FIG. 1 is a schematic view of one embodiment of a machine for administering gas to a patient comprising an NO delivery apparatus according to the invention;

FIG. 2 is a schematic representation of gas-analysing means of an NO delivery apparatus according to the invention, such as that of the machine of FIG. 1;

FIG. 3 schematically shows one embodiment of displays effected on the display screen of an NO delivery apparatus according to the invention, such as that of the machine of FIG. 1.

### DETAILED DESCRIPTION

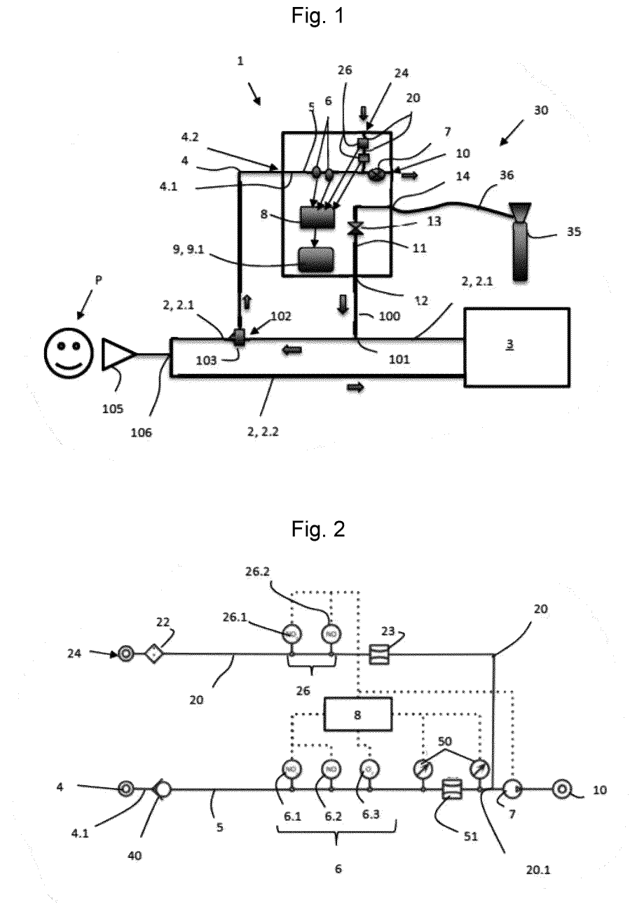

FIG. 1 schematically shows one embodiment of a gas delivery machine 30 allowing an NO-containing gas mixture to be supplied to a patient having need therefor, comprising an NO delivery device or apparatus 1 for supplying an NO-based gas, typically an NO/N₂ gas mixture, and a medical ventilator 3, i.e. an apparatus for assisting with breathing that delivers a respiratory gas containing oxygen, typically at least about 20% oxygen (O₂), and in general at least about 21% oxygen.

Such a machine 30 makes it possible to deliver, to a patient P, a final gas mixture containing NO in a desired concentration corresponding to a dosage set by an anaesthetist or the like, typically between 1 and 80 ppmv of NO (i.e. ppm by volume). The final gas mixture is formed by mixing an NO-based flow of gas, such as an NO/N₂ mixture coming from the NO delivery apparatus 1, and a flow of respiratory gas containing oxygen, typically at least about 20% oxygen (O₂), such as air or an O₂/N₂ mixture, coming from the medical ventilator 3.

The flow of NO-based gas (i.e. NO/N₂), which exits the NO delivery apparatus 1 via an outlet 12, is transported by a flexible conduit 100 and is then injected (at 101) into a patient circuit 2, and in particular into the inhalation branch 2.1 of the patient circuit 2, which is moreover fed with the oxygen-containing gas flow supplied by the medical ventilator 3. The patient circuit 2 for example comprises flexible pipes, for example flexible pipes made of polymer.

The flow rate of the O₂-based gas flow flowing through the patient circuit 2 is measured by a primary flow-rate sensor (not shown) arranged in the patient circuit 2, i.e. in the inhalation branch 2.1, which sensor is electrically connected to the control means 8 of the NO delivery apparatus 1 in order to supply them with flow-rate measurements or signals reflecting the flow rate of the oxygen-containing gas supplied by the medical ventilator 3 and flowing through the patient circuit 2 in the direction of the patient P. The primary flow-rate sensor may be a mass flow meter, a pressure differential flow meter, or the like.

The final gas mixture obtained by mixing the flows of NO/N₂ and of air or an O₂/N₂ mixture is mainly formed of oxygen and nitrogen, and of NO with the desired dosage, typically between 1 and 80 ppmv of NO; it may contain unavoidable impurities, for example argon or the like. It is administered to the patient P, when he inhales, by means of a respiratory interface 105, for example a respiratory mask, a tracheal tube or any other suitable interface.

The gases exhaled by the patient when he exhales are collected by an exhalation branch 2.2 of the patient circuit 2. The inhalation and exhalation branches 2.1, 2.2 comprise conduits, ducts, pipes, passages, tubing or the like, for example flexible polymer pipes, that are able and configured to transmit gas flows, and that are fluidically connected to a junction part 106, such as a Y-shape part or the like, that is moreover connected to the respiratory interface 105. The respiratory interface 105 makes it possible to deliver the gas to the patient P, in particular when he inhales, and to collect the (CO₂-enriched) gases exhaled by the patient P, in particular when he exhales.

During operation of the machine 30, the gas flow flowing through the inhalation branch 2.1 of the patient circuit 2, i.e. travelling from the mechanical ventilator 3 to the patient P, is inhaled by the patient, while the gases exhaled thereby, i.e. gases enriched with CO₂, are transmitted by the exhalation branch 2.1 to the ventilator 3, where they are exhausted to atmosphere via a venting orifice (not shown) or the like.

Generally, the NO delivery apparatus 1 has a conventional architecture and operates in a conventional way, as regards the supply of NO, typically an NO/N₂ mixture. It comprises (not shown) an internal primary gas circuit 11 for carrying the NO-containing gas, in particular an (or more than one) internal line(s) for transporting gas, typically an NO/N₂ mixture containing from 100 to 2000 ppmv of NO (remainder N₂), and typically less than 1000 ppmv of NO.

Means 13 for controlling flow rate, such as a solenoid valve, and/or for controlling the pressure of the gas flow, such as a gas expansion device, are arranged in the primary gas circuit 11. The means 13 for controlling flow rate may be controlled by the control means 8 of the NO delivery apparatus 1, particularly on the basis of the flow-rate measurements or signals supplied by the primary flow-rate sensor.

The control means 8 comprise control electronics, typically one or more (micro)processors or microcontrollers borne by a (or more than one) circuit board(s) and implementing one or more algorithms configured to process the measurements or other information, data or the like.

The primary flow-rate sensor is advantageously arranged in the inhalation branch 2.1, upstream of the injection site 101 where NO (i.e. the NO/nitrogen mixture) is injected into the inhalation branch 2.1 and mixed with the oxygen-containing gas flow coming from the medical ventilator 3.

The NO/N₂ mixture passing through the NO delivery apparatus 1 comes from one or more gas sources 35, typically one or more cylinders of compressed gas (not shown) containing the NO/N₂ gas mixture at a pressure of up to 150 bar, or even 180 to 200 bar, or more. The gas source(s) 35 fluidically connect, via a (or more than one) flexible conduit(s) 36, to (at least) a gas inlet 14 of the primary gas circuit 11 of the NO delivery device 1.

As already explained, the NO delivery apparatus 1 makes it possible to supply/inject (at 101) the NO-based (i.e. NO/nitrogen) mixture into the patient circuit 2 so that it mixes there with (i.e. is diluted by) the oxygen-containing gas flow coming from the medical ventilator 2, which for example is an N₂/O₂ mixture or air.

The final combined NO/O₂/N₂ gas mixture, which is then administered to the patient P via the respiratory interface 105, mainly contains nitrogen (N₂), oxygen (O₂) in a content of at least about 20 vol. %, and NO in a content between 1 and 80 ppmv, and typically of the order of 5 to 40 ppmv, corresponding to the desired dosage, which particularly depends on the population treated, i.e. newborns, children, adolescents or adults, and on the disease to be treated.

Specifically, nitric oxide or NO, when inhaled, dilates the pulmonary vessels and increases oxygenation by improving gas exchange. As particularly described in EP-A-560928, EP-A-1516639 and US-A-10,201,564, these properties are used to treat various medical conditions, such as persistent pulmonary hypertension of the newborn (PPHN); acute respiratory distress syndrome (ARDS), which is mainly observed in adults; or post cardiac surgery pulmonary hypertension (PH) in adults or children.

However, on account of the presence of O₂ in the final NO/O₂/N₂ gas mixture, some of the NO present therein may oxidize and form toxic NO₂ species.

It is therefore essential to ensure that the NO concentration in the final NO/O₂/N₂ gas mixture does indeed correspond to the dosage desired by the physician and that, moreover, the amount of toxic NO₂ species is limited, typically to less than a few ppmv, and in general to less than 0.5 ppmv.

To do this, the NO delivery apparatus 1 of FIG. 1 incorporates gas-analysing means 5, 6, i.e. a gas analyser, that are arranged in the outer carcass or shell, which for example is made of polymer, of the NO delivery apparatus 1, and that allow the NO and NO₂ concentrations in the final gas mixture to be monitored, i.e. measured, as explained below.

As detailed in FIG. 2, the gas-analysing means 5, 6 comprise a primary gas-analysis line 5 comprising first concentration-measuring means 6 configured to take measurements of NO and/or NO₂, or even also oxygen, content in the gas flow present within said primary gas-analysis line 5.

A gas-sampling line 4, such as a flexible pipe, fluidically connects the patient circuit 2 to the primary gas-analysis line 5 so as to allow gas samples to be drawn from the circuit 2, i.e. from the inhalation branch 2.1, and transported to the primary gas-analysis line 5, in order to bring them into contact with the first concentration-measuring means 6, so that they may take measurements of NO and NO₂ and/or O₂ contents.

Preferably, the concentration-measuring means 6 comprise NO, NO₂ and possibly O₂ sensors, for example electrochemical sensors or the like. The sensors may each be dedicated to one given compound (NO, NO₂ or O₂) or may be combined to measure the contents of a plurality of compounds, in particular NO and NO₂.

The concentration measurements taken by the first concentration-measuring means 6 are transmitted to the control means 8, i.e. to the control electronics, which process them in order to deduce therefrom values of the NO and NO₂ and/or O₂ contents.

In order to allow gas, i.e. gas samples, to be drawn from the circuit 2, gas-pumping means 7, such as a suction pump, are arranged in the primary gas-analysis line 5, preferably downstream of the first concentration-measuring means 6, so as to pump up these gas samples. The suction pump may be a diaphragm or piezoelectric suction pump.

As illustrated in FIG. 2, the primary gas-analysis line 5 may also comprise pressure taps or sensors 50 arranged on either side of a device 51 with a calibrated orifice, particularly so as to form a differential pressure sensor, the pressure sensors or taps being electrically connected to the control means 8, which may be configured to determine a gas flow rate from the differential pressure measurements.

Moreover, the NO delivery apparatus 1 also comprises display means 9 for displaying various pieces of information, values, data, alarms, graphics, icons, windows, curves, gauges or the like. Preferably, they comprise a display screen 9.1, preferably a touch-sensitive screen, i.e. a screen with a touch panel. The displays are preferably in colour.

In particular, the touch-sensitive display screen 9.1 also makes it possible to display one or more virtual keys (i.e. predefined areas) that are actuated by touch, particularly in order to effect one or more activations, selections or validations, for example in selection menus or in virtual windows also displayed on the screen 9.1. A (or more than one) such virtual key(s) are actuatable by the user through a press on the panel/cover glass of the screen 9.1, i.e. activatable or selectable by the user, i.e. the caregiver, via digital contact or pressure applied with a finger.

Thus, the display screen 9.1 also makes it possible to display the NO and/or NO₂, and possibly O₂, contents determined by the control means 8 from the measurements taken by the first concentration-measuring means 6, and typically by the NO, NO₂ and possibly O₂ sensors. Such display allows the caregiver to ensure that the final, e.g. (NO/N₂/O₂), gas mixture contains the desired dosage of NO, a non-hypoxic oxygen content (i.e. >20 vol. %) and, conversely, does not contain an excessive amount of toxic NO₂ species.

The control means 8, in particular the microprocessor(s) or microcontroller, are also configured to control all or some of the displays that are effected on the display screen 9.1, i.e. on the display means, in particular the values of the determined NO and NO₂ contents.

The electrical supply to the NO delivery apparatus 1, in particular to the control means 8, the sensors, the pump 7, the screen 9.1, etc., is provided by an electrical current source and/or electrical supply means (not shown), for example a connection to (110/220 V) mains current (such as an electrical cable and plug) and/or one or more, preferably rechargeable, electric batteries, and/or a current transformer.

The apparatus 1 may also comprise storage means, such as a flash memory or the like. It may be integrated into the control means 8, and for example borne by a circuit board.

In the embodiment of FIG. 1, the gas-sampling line 4, such as a flexible pipe, is connected, via its downstream end, to the primary gas-analysis line 5 via a linking section 4.1 located in the apparatus 1. The linking section 4.1 comprises an inlet orifice 4.2 to which the primary gas-sampling line 4 is connected, for example the inlet orifice 4.2 may be borne by a fluidic fastening or connector or the like. According to another embodiment (not shown), the primary gas-sampling line 4 may be connected directly to the gas-analysis line 5.

Moreover, the upstream end of the primary gas-sampling line 4 is connected to the patient circuit 2 (at 102) via a suitable connecting device 103, for example a T-shaped fluidic connector or the like arranged downstream of the point 101 of injection of NO into the patient circuit.

The gas-pumping means 7, typically a suction pump, allows the gas to be made to flow through the gas-sampling line 4 and into the primary gas-analysis line 5, here via the section 4.1, through creation of a negative pressure that generates a gas flow or stream that will course through the first means 6 for measuring NO and NO₂ (or even also O₂) concentration, i.e. the sensors of the primary gas-analysis line 5, so that said sensors may take the desired concentration measurements.

The pumping rate is preferably between 50 and 350 mL/min, and for example of the order of 200 to 250 mL/min.

A non-return device 40 arranged in the section 4.1 or in the primary gas-analysis line 5 makes it possible to prevent any return of the gas flow to the patient circuit 2.

The gas flow to be analysed is therefore pumped by the pump of the gas-pumping means 7 and flows through the primary gas-analysis line 5, so as to bring it into contact with the NO₂ and NO sensors, which will then take a measurement of the NO₂ and NO concentration of the gas flow and supply these measurements to the control means 8, which will then render them to the user, such as a physician or another caregiver, by commanding their display on the screen 9.1, which may form part of a graphical user interface (GUI) of the NO delivery apparatus 1.

The primary gas-analysis line 110 may also comprise one or more other sensors, in particular an oxygen sensor, and the oxygen concentration may also be displayed on the screen 9.1 in order to allow the user to monitor it and ensure that the gas mixture supplied to the patient is not hypoxic or toxic.

Of course, if necessary, the control means 8 may also process the NO and NO₂ measurements before commanding their display on the screen 9.1, in particular carry out a compensation of these values to take account of environmental factors which may influence the measurements, such as atmospheric pressure, temperature and/or humidity, and to which the gas mixture may have been subjected.

Advantageously, the control means 8 comprise a (or more than one) control circuit board(s) and a control unit with one or more (micro)processors or microcontrollers, or the like. Preferably, the control means 8 also command, i.e. control, the suction pump 7 in such a way that the flow rate of the flow flowing through the primary gas-analysis line 5 is kept constant and equal to the desired target flow rate, e.g. between 50 and 350 mL/min.

After analysis, the analysed gas flow is discharged to atmosphere after having transited through the primary gas-analysis line 5 and through the suction pump 7 located between the first concentration-measuring means 6, i.e. the NO and NO₂ sensors 6.1, 6.2 (or even also the O₂ sensor 6.3), and an outlet orifice or vent 10 in fluidic communication with ambient air, i.e. the atmosphere of the room in which the apparatus 1 is located.

The ambient air is therefore ‘polluted’ with these NO and NO₂ compounds and those released in other manners, for example during purges of flexible pipes or the like.

Now, some caregivers, mainly paramedics, fear being exposed to NO and NO₂ while providing care in the room where the patient or patients are treated through administration of NO, for example in a treatment or resuscitation room, making them anxious and potentially affecting their health.

Therefore, in order to attempt a remedy, according to the invention, the NO delivery apparatus has been improved in such a way as to be able to determine and display the concentration of NO and/or NO₂ compounds in the ambient air of the setting where the NO-supplying machine 30 is located and/or used, e.g. a treatment room, resuscitation room, a vehicle interior, or the like.

Specifically, knowing their level of exposure to the NO and NO₂ compounds present in the ambient air allows caregivers' anxiety to be decreased and moreover their health to be protected by making it possible to ensure that they are not subjected to excessive doses, typically ones greater than OELs.

To do this, according to the invention, as shown in FIG. 1 and detailed in FIG. 2, provision has been made, in the NO delivery apparatus 1, for a secondary gas-analysis line 20 that fluidically communicates with ambient atmosphere through an ambient-air inlet 24.

The secondary gas-analysis line 20 is fluidically connected to the primary gas-analysis line 5 at a connection site 20.1 located upstream of the pumping means 7, i.e. of the pump, so that the pumping means 7 may also pump and make ambient air flow through the secondary gas-analysis line 20.

As detailed in FIG. 2, the secondary gas-analysis line 20 comprises second concentration-measuring means 26 allowing, as explained above, measurements of the NO and/or NO₂ content in the ambient-air flow present and flowing within the secondary gas-analysis line 20 to be taken, typically an NO sensor 26.1 and/or an NO₂ sensor 26.2. These sensors may be of the same type as those of the primary gas-analysis line 5 or other types of sensor.

Typically, the site 20.1 of connection of the secondary gas-analysis line 20 to the primary gas-analysis line 5 is located between the NO and NO₂ sensors 6.1, 6.2 and the pumping means 7.

Again, the NO and NO₂ concentration measurements taken by the second concentration-measuring means 26 are supplied to the control means 8, which process them to determine the NO and/or NO₂ contents measured by the second concentration-measuring means 26, and then to control the display means 9 to display these NO and/or NO₂ contents.

In the embodiment of FIG. 2, the secondary gas-analysis line 20 is in constant fluidic communication with the primary gas-analysis line 5 and hence the pumping means 7 may pump gas from the two gas-analysis lines 5, 20 simultaneously and their analysis may be carried out concomitantly, i.e. at the same time, and constantly.

The air flow to be analysed is therefore pumped by the pump of the gas-pumping means 7 and flows through the secondary gas-analysis line 20, so as to bring it into contact with the second NO₂ and NO sensors 26.1, 26.2, which will take measurements of the NO₂ and NO concentration of the air flow and supply these measurements to the control means 8, which will then process them and render them to the user, such as a physician or another caregiver, by commanding their display on the screen 9.1, as explained above.

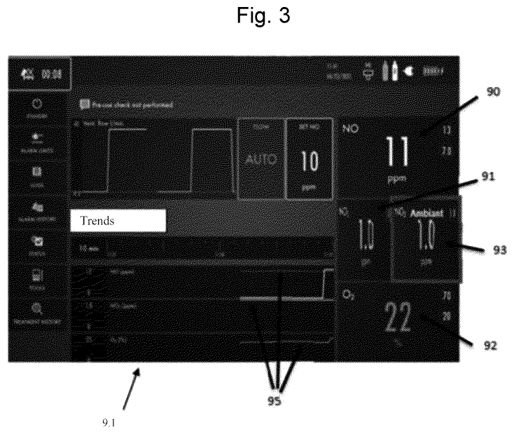

Advantageously, the screen 9.1 displays, simultaneously and constantly, the NO₂ and/or NO concentrations measured in the primary and secondary gas-analysis lines 5, 20, in order to constantly apprise the user of the NO₂ and/or NO concentrations not only in the combined gas mixture administered to the patient but also in the ambient atmosphere surrounding the apparatus, for example in the air of the room or passenger compartment where the NO delivery apparatus 1 is located, as for example illustrated in FIG. 3.

Moreover, as illustrated in FIG. 2, it may be seen that the secondary gas-analysis line 20 preferably comprises a filtering means 22, such as a HEPA filter or the like, located downstream of the air inlet 24, serving to purify the air by removing dust, spores, bacteria or other aerosols that are liable to be found therein; and/or a device 23 with a calibrated orifice, arranged between the NO₂ and NO sensors 26.1, 26.2 and the site 20.1 of connection to the primary analysis line 5, which makes it possible to generate (when necessary) a head loss in the air flow through the secondary analysis line 20 equivalent to the head loss occurring in the sampling line 4, for example when the latter is equipped, for example, with a device such as a “water trap” (not shown). Specifically, such “balancing” allows the pump 7 to remain at the same speed when it is pumping gas coming from the primary gas-analysis line 5 or from the secondary gas-analysis line 20.

In other words, according to the invention, the secondary gas-analysis line 20 and the primary gas-analysis line 5 are advantageously in constant fluidic communication with the pumping means 7, i.e. the pump, and hence the pumping means 7 may pump and make flow, at the same time, ambient air through the secondary gas-analysis line 20 and the combined gas mixture coming from the patient circuit 2, in order to make it possible to determine and then display simultaneously the NO and NO₂ contents in these two gas flows.

Generally, as illustrated in FIG. 3, once measurements have been taken by the first and second NO sensors 6.1, 26.1 and NO₂ sensors 6.2, 26.2, and preferably also by the O₂ sensor 6.3, and transmitted to the control means 8, they are processed by the latter, which moreover control their simultaneous display on the screen 9.1, during treatment of the patient P, this allowing the caregiver to reassure himself them that the composition of the final gas supplied to the patient is indeed compliant with the expected dosage and does not contain toxic NO₂ species in amounts above the maximum allowable threshold(s).

Thus, in FIG. 3, the following are simultaneously displayed on screen 9.1: the concentrations of NO (ref. no. 90), NO₂ (ref. no. 91) and O₂ (ref. no. 92) measured by the first NO sensor 6.1, NO₂ sensor 6.2 and O₂ sensor 6.3 in the combined gas flow supplied to the patient, and the NO₂ content measured by the second NO₂ sensor 26.1 (ref. no. 93) in the ambient-air flow. Here, the NO content in the air is not displayed, but it could also be.

It will moreover be noted that trend curves for the NO, NO₂ and O₂ concentrations (ref. no. 95) are also displayed, as is other information, particularly alarm thresholds for the various compounds (NO, NO₂, O₂), and the setting for NO content in the combined gas flow sent to the patient (i.e. dosage).

An audible and/or visual alarm may be triggered in case of detection of an excessive NO₂ concentration and/or of an excessive or insufficient NO and/or O₂ concentration.

Generally, the gas-administrating machine 30 comprising the NO delivery apparatus 1 according to the invention is suitable for use in treating various pulmonary pathologies, such as persistent pulmonary hypertension of the newborn (PPHN), acute respiratory distress syndrome (ARDS) and/or post cardiac surgery pulmonary hypertension (PH), in different patient populations, and in particular newborns, adults, adolescents or children.

What is claimed is:

1. NO delivery apparatus (1) comprising:
a primary gas circuit (11) for carrying an NO-containing gas to at least one gas outlet (12) configured to be fluidically connected to a patient circuit (2) carrying an oxygen-containing respiratory gas,
a primary gas-analysis line (5) comprising first concentration-measuring means (6) configured to take measurements of NO and/or NO₂ content in the gas flow present within said primary gas-analysis line (5),
gas-pumping means (7) configured to pump gas and make it flow through at least the primary gas-analysis line (5),
control means (8) configured to process the concentration measurements taken by the concentration-measuring means (6) and to deduce therefrom at least one NO and/or NO₂ content, and
display means (9) configured to display said at least one NO and/or NO₂ content measured by said first concentration-measuring means (6), characterized in that:
it further comprises a secondary gas-analysis line (20) fluidically communicating with ambient atmosphere,
the gas-pumping means (7) are configured and/or arranged to pump ambient air and make it flow through the secondary gas-analysis line (20),
the secondary gas-analysis line (20) comprises second concentration-measuring means (26) configured to take measurements of NO and/or NO₂ content in the ambient-air flow present within said secondary gas-analysis line (20),
the control means (8) are further configured to process the concentration measurements taken by the second concentration-measuring means (26), and
the display means (9) are further configured to display said at least one NO and/or NO₂ content measured by said second concentration-measuring means (26).

2. Apparatus according to claim 1, characterized in that the secondary gas-analysis line (20) is fluidically connected (20.1) to the primary gas-analysis line (5), preferably between the first concentration-measuring means (6) and the gas-pumping means (7).

3. Apparatus according claim 1, characterized in that the secondary gas-analysis line (20) and the primary gas-analysis line (5) are in permanent fluidic communication with each other so that the gas-pumping means (7) simultaneously pump gas from said primary and secondary gas-analysis lines (5, 20).

4. Apparatus according to claim 1, characterized in that the gas-pumping means (7) comprise a suction pump comprising a gas inlet fluidically connected to the primary gas-analysis line (5) and/or to the secondary gas-analysis line (20), and a gas outlet fluidically connected to atmosphere, preferably via an outlet orifice (10).

5. Apparatus according to claim 1, characterized in that the control means (8) are configured to control the gas-pumping means (7) to pump gas, preferably at a given pumping rate between 50 and 350 mL/min.

6. Apparatus according to claim 1, characterized in that the first and second concentration-measuring means (6; 26) comprise a first and a second NO sensor (6.1; 26.1) and/or a first and a second NO₂ sensor (6.2; 26.2).

7. Apparatus according to claim 1, characterized in that the display means (9) comprise a touch-sensitive display screen (9.1).

8. Apparatus according to claim 1, characterized in that the control means (8) comprise control electronics.

9. Apparatus according to claim 1, characterized in that the touch-sensitive screen (9.1) is configured to display (95) simultaneously the NO content and/or NO₂ content determined by the first and second concentration-measuring means (6; 26).

10. Machine for administering gas (100) to a patient (P) comprising: the NO delivery apparatus (1) according to claim 1; a medical ventilator (3); and the patient circuit (2) to which are fluidically connected the NO delivery apparatus (1) and the medical ventilator (3), and wherein the patient circuit (2) is fluidically connected to the primary gas-analysis line (5) via a gas-sampling line (4).
